(19) United States
(12) Patent Application Publication
(10) Pub. No.: US 2024/0361337 A1
(43) Pub. Date: Oct. 31, 2024

(54) BIOMARKERS FOR EARLY DIAGNOSIS OF CHRONIC HIP DISPLASIA AND METHODS OF USING THE SAME

(71) Applicant: The Curators of the University of Missouri, Columbia, MO (US)

(72) Inventors: James L. Cook, Columbia, MO (US); Aaron M. Stoker, Columbia, MO (US); Chantelle C. Bozynski, Columbia, MO (US); Keiichi Kuroki, Columbia, MO (US); Cristi R. Cook, Columbia, MO (US); Emily Leary, Columbia, MO (US)

(21) Appl. No.: 18/660,434

(22) Filed: May 10, 2024

Related U.S. Application Data
(63) Continuation of application No. 17/168,871, filed on Feb. 5, 2021, now abandoned.
(60) Provisional application No. 62/970,579, filed on Feb. 5, 2020.

(51) Int. Cl. G01N 33/68

(52) U.S. Cl. CPC G01N 33/6893; G01N 2800/60

(57) ABSTRACT

Methods for predicting canine hip dysplasia (CHD) in an immature canine subject are provided. The methods include the use of concentration profiles of a plurality of polypeptides, including C2C, CTX-I, CTX-II, RANKL, PIICP, COMP, PINP. Also provided are CHD biomarker profiles for predicting or diagnosing CHD in immature or young canines before CHD develops. Diagnostic reagents and kits for the same are also provided.

### CROSS-REFERENCE TO RELATED APPLICATION

This application is a continuation of U.S. patent application Ser. No. 17/168,871 filed on Feb. 5, 2021, which claims benefit of and priority to U.S. Provisional Patent Application Ser. No. 62/970,579, filed Feb. 5, 2020, herein incorporated by reference in its entirety.

### TECHNICAL FIELD

The presently disclosed subject matter is directed to biomarkers for early diagnosis of chronic hip dysplasia and methods of using the same. Also provided are diagnostic and/or predictive kits and assays for chronic hip dysplasia and related conditions in canines.

### BACKGROUND

Canine hip dysplasia (CHD) is a multi-factorial, developmental condition that has a polygenic mode of inheritance, characterized by joint laxity and abnormal development of the femoral head and acetabulum similar to various dysplasias of the hip seen in people. CHD is a common cause of secondary osteoarthritis (OA) in affected dogs. Secondary OA is a profound health problem in dogs with associated costs of billions of dollars to the pet-owning public, which mimics the situation in human healthcare. According to data from the Orthopedic Foundation for Animals (OFA), the prevalence of hip dysplasia can be as high as 72% in some breeds. Currently, definitive diagnosis is based on radiographic evaluation, which is typically performed after the potentially reversible stage of the disorder. Even then, breeding selection based on radiographic evaluation has led to only a modest reduction in CHD prevalence.

Thus, there is a need for a more optimal method of diagnosis for canine hip dysplasia. What is needed are protein biomarkers with high discriminatory capabilities for differentiating dysplastic dogs from dogs without hip dysplasia. More particularly, what is needed is the development and validation of a panel of biomarkers in urine and/or serum to discriminate puppies with dysplastic hips from normal puppies early in development (i.e., prior to skeletal maturity [<1 year of age]).

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

In some embodiments, provided herein are methods for diagnosing and/or predicting canine hip dysplasia (CHD) in an immature canine subject. The methods can comprise obtaining a biological sample from an immature canine subject, measuring in the biological sample from the immature canine subject a concentration of at least two polypeptides selected from the group consisting of: C2C, CTX-I, CTX-II, RANKL, PIICP, COMP, PINP; generating a sample polypeptide concentration profile based on the concentration of the at least two polypeptides in the biological sample, wherein an elevated level of at least two polypeptides in the biological sample, as compared to a control, provides a sample polypeptide profile indicative of CHD, or a susceptibility to CHD, wherein an unelevated level of the at least two polypeptides in the biological sample, as compared to a control, provides a sample polypeptide profile indicative of no CHD, or no susceptibility to CHD, and using the generated sample polypeptide profile to diagnose and/or predict CHD in the immature canine subject.

In some embodiments of such methods the immature canine subject is a canine that has not reached skeletal maturity or is less than about two years of age. Optionally, in some aspects the immature canine subject is a canine that is less than about six months of age.

In some embodiments of such methods the biological sample comprises any one of urine, whole blood, blood plasma, synovial fluid and serum. In some embodiments, the biological sample comprises urine.

In some embodiments, the concentration of at least three polypeptides selected from the group consisting of C2C, CTX-I, CTX-II, RANKL, PIICP, COMP and PINP is measured, and wherein the sample polypeptide profile is based on the concentration of the at least three polypeptides in the biological sample. In some embodiments, an elevated or decreased (or combination thereof) concentration of at least two of the three polypeptides in the biological sample, as compared to a control, provides a sample polypeptide profile indicative of CHD. In some embodiments, an elevated or decreased (or combination thereof) concentration of the three polypeptides in the biological sample, as compared to a control, provides a sample polypeptide profile indicative of CHD. In some embodiments, the concentration of at least four polypeptides selected from the group consisting of C2C, CTX-I, CTX-II, RANKL, PIICP, COMP and PINP is measured, and wherein the sample polypeptide profile is based on the at least four polypeptides in the biological sample. In some embodiments, an elevated or decreased (or combination thereof) concentration of at least three of the four polypeptides in the biological sample, as compared to a control, provides a sample polypeptide profile indicative of CHD. In some embodiments, an elevated or decreased (or combination thereof) concentration of the four polypeptides in the biological sample, as compared to a control, provides a sample polypeptide profile indicative of. In some embodiments, the concentration of each of C2C, CTX-I, CTX-II and RANKL is measured in a urine sample from the immature canine subject, and wherein the sample polypeptide profile is based on each of C2C, CTX-I, CTX-II and RANKL in the urine sample. In some embodiments, the concentration of each of C2C, COMP, PIICP, CTX-II and RANKL is measured in a serum sample from the immature canine subject, and wherein the sample polypeptide profile is based on each of C2C, COMP, PIICP, CTX-II and RANKL in the serum sample.

In some embodiments, the concentration of the at least two polypeptides in the biological sample is measured using a method selected from the group consisting of: LUMINEX, ELISA, immunoassay, mass spectrometry, high performance liquid chromatography, two-dimensional electrophoresis, qPCR, RT-PCR, nucleic acid microarray, in situ hybridization, SAGE, Western blotting, protein microarray, and antibody microarray.

Also provided herein are canine hip dysplasia (CHD) biomarker concentration profiles comprising polypeptide concentration levels for two or more polypeptides selected from the group consisting of: C2C, CTX-I, CTX-II, RANKL, PIICP, COMP, PINP, and fragments of any thereof, and any combination thereof obtained from a biological sample from at least one immature canine subject susceptible to having and/or developing CHD. In some embodiments, the CHD biomarker profile can further comprise polypeptide levels for at least one biological sample obtained from at least one healthy canine subject and/or a canine subject not susceptible to developing CHD. In some embodiments, the biological samples from the immature canine subject susceptible to having and/or developing CHD and the healthy subject and/or the subject not susceptible to developing CHD both comprise a sample selected from the group consisting of urine, whole blood, blood plasma, synovial fluid and serum. In some embodiments, the biological samples are urine samples.

In some embodiments, the CHD biomarker concentration profiles can comprise polypeptide concentration levels for three or more polypeptides selected from the group consisting of C2C, CTX-I, CTX-II, RANKL, PIICP, COMP, PINP, optionally for four or more polypeptides selected from the group consisting of C2C, CTX-I, CTX-II, RANKL, PIICP, COMP, PINP. In some embodiments, the CHD biomarker concentration profile can comprise polypeptide concentration levels of each of C2C, CTX-I, CTX-II and RANKL, wherein the sample is a urine sample, or optionally polypeptide concentration levels of each of C2C, COMP, PIICP, CTX-II and RANKL, wherein the sample is a serum sample.

In some aspects, provided herein are diagnostic reagents for diagnosing and/or predicting canine hip dysplasia (CHD) comprising at least one antibody against at least one CHD biomarker or fragment thereof selected from the group consisting of: C2C, CTX-I, CTX-II, RANKL, PIICP, COMP, PINP. In some embodiments, such diagnostic reagents can comprise at least two or more antibodies against any two or more CHD biomarkers or fragments thereof. In some aspects a kit comprising the diagnostic reagents is provided.

Provided herein in some embodiments are kits for diagnosing and/or predicting canine hip dysplasia (CHD) in an immature canine subject. Such kits can in some aspects comprise at least one CHD biomarker detection reagent that specifically binds to a CHD polypeptide selected from the group consisting of C2C, CTX-I, CTX-II, RANKL, PIICP, COMP, PINP and fragments of any thereof, or at least one CHD biomarker detection reagent that specifically binds to at least part of a polynucleotide sequence coding for at least one of the CHD polypeptides, wherein the specific binding of the reagent is indicative of the production level of at least one of the CHD polypeptides in a biological sample from a subject. Such kits can further comprise at least two CHD biomarker detection reagents that each specifically binds to a CHD polypeptide, or at least two CHD biomarker detection reagents that each specifically binds to at least part of a polynucleotide sequence coding for at least one of the CHD polypeptides. The reagents that specifically detect production of at least one biomarker can comprise a nucleic acid probe complementary to at least part of a polynucleotide sequence coding for one of the polypeptides. The nucleic acid probe can be a cDNA or an oligonucleotide. In some aspects, at least one CHD biomarker detection reagent can be immobilized on a substrate surface, or optionally at least two biomarker detection reagents can be arranged on the substrate surface. In some embodiments, at least two biomarker reagents can be arranged on the substrate surface comprise a microarray.

These and other objects are achieved in whole or in part by the presently disclosed subject matter. Further, objects of the presently disclosed subject matter having been stated above, other objects and advantages of the presently disclosed subject matter will become apparent to those skilled in the art after a study of the following description, Figures and Examples.

### BRIEF DESCRIPTION OF THE FIGURES

The presently disclosed subject matter can be better understood by referring to the following figures. The components in the figures are not necessarily to scale, emphasis instead being placed upon illustrating the principles of the presently disclosed subject matter (often schematically). The figures are not intended to limit the scope of this presently disclosed subject matter, which is set forth with particularity in the claims as appended or as subsequently amended, but merely to clarify and exemplify the presently disclosed subject matter.

For a more complete understanding of the presently disclosed subject matter, reference is now made to the following figures in which:

FIG. 1 is a graphical depiction of data based on the measurement of the urine collagenase-generated cleavage epitope of type II collagen (C2C) concentration (ng/mL) over time (time points in months age) in immature (less than 12 months of age) and mature canines, as measured using an enzyme-linked immunosorbent assay (ELISA). At 5 months of age, there is a significant (p<0.05) difference in concentration of C2C between canines with normal and dysplastic hips.

FIG. 2 is a graphical depiction of data based on the measurement of the urine cartilage oligomeric matrix protein (COMP) concentration (ng/mL) over time (time points in months of age) in immature (less than 12 months of age) and mature canines with normal and dysplastic hips, as measured using an ELISA.

FIG. 3 is a graphical depiction of data based on the measurement of the urine cross linked C-telopeptide of type I collagen (CTX-I) concentration (ng/mL) over time (time points in months of age) in immature (less than 12 months of age) and mature canines with normal and dysplastic hips, as measured using an ELISA. At 5 months of age, there is a marked difference in concentration of CTX-1 between canines with normal and dysplastic hips.

FIG. 4 is a graphical depiction of data based on the measurement of the urine cross linked C-telopeptide of type II collagen (CTX-II) concentration (pg/mL) over time (time points in months of age) in immature (less than 12 months of age) and mature canines with normal and dysplastic hips, as measured using an ELISA. At 5 months of age, there is a marked difference in concentration of CTX-II between canines with normal and dysplastic hips.

FIG. 5 is a graphical depiction of data based on the measurement of the urine procollagen type II C-terminal propeptide (PIICP) concentration (ng/mL) over time (time points in months of age) in immature (less than 12 months of age) and mature canines with normal and dysplastic hips, as measured using an ELISA.

FIG. 6 is a graphical depiction of data based on the measurement of the urine procollagen type I N-terminal propeptide (PINP) concentration (pg/mL) over time (time points in months of age) in immature (less than 12 months of age) and mature canines with normal and dysplastic hips, as measured using an ELISA.

FIG. 7 is a graphical depiction of data based on the measurement of the urine receptor activator of nuclear factor kappa-B ligand (RANKL) concentration (ng/mL) over time (time points in months of age) in immature (less than 12 months of age) and mature canines with normal and dysplastic hips, as measured using an ELISA. At 5 months of age, there is a significant (p<0.05) difference in concentration of RANKL between canines with normal and dysplastic hips.

FIG. 8 is a graphical depiction of log-transformed concentrations (ng/ml) of selected urine biomarkers (C2C, CTX-II, CTX-I, and RANKL) obtained from immature canines with or without hip dysplasia at 5 months of age. Immature canines with dysplastic hips have higher levels of all urine biomarkers compared to immature canines with normal hips.

FIG. 9 is a graphical depiction of data based on the measurement of the serum collagenase-generated cleavage epitope of type II collagen (C2C) concentration (ng/mL) over time (time points in months of age) in immature (less than 12 months of age) and mature canines with normal and dysplastic hips, as measured using an enzyme-linked immunosorbent assay (ELISA).

FIG. 10 is a graphical depiction of data based on the measurement of the serum cartilage oligomeric matrix protein (COMP) concentration (ng/mL) over time (time points in months of age) in immature (less than 12 months of age) and mature canines with normal and dysplastic hips, as measured using an ELISA.

FIG. 11 is a graphical depiction of data based on the measurement of the urine cross linked C-telopeptide of type I collagen (CTX-I) concentration (ng/mL) over time (time points in months of age) in immature (less than 12 months of age) and mature canines with normal and dysplastic hips, as measured using an ELISA.

FIG. 12 is a graphical depiction of data based on the measurement of the urine cross linked C-telopeptide of type II collagen (CTX-II) concentration (pg/mL) over time (time points in months of age) in immature (less than 12 months of age) and mature canines with normal and dysplastic hips, as measured using an ELISA.

FIG. 13 is a graphical depiction of data based on the measurement of the urine procollagen type II C-terminal propeptide (PIICP) concentration (ng/mL) over time (time points in months of age) in immature (less than 12 months of age) and mature canines with normal and dysplastic hips, as measured using an ELISA. At all time points (including at 5 months of age), there are marked differences in concentration of PIICP between canines with normal and dysplastic hips.

FIG. 14 is a graphical depiction of data based on the measurement of the urine procollagen type I N-terminal propeptide (PINP) concentration (pg/mL) over time (time points in months of age) in immature (less than 12 months of age) and mature canines with normal and dysplastic hips, as measured using an ELISA.

FIG. 15 is a graphical depiction of data based on the measurement of the urine receptor activator of nuclear factor kappa-B ligand (RANKL) concentration (ng/mL) over time (time points in months of age) in immature (less than 12 months of age) and mature canines with normal and dysplastic hips, as measured using an ELISA.

### DETAILED DESCRIPTION

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

### I. Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of a composition, dose, sequence identity (e.g., when comparing two or more nucleotide or amino acid sequences), mass, weight, temperature, time, volume, concentration, percentage, etc., is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

The term “gene” refers broadly to any segment of DNA associated with a biological function. A gene can comprise sequences including but not limited to a coding sequence, a promoter region, a cis-regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation of an existing sequence.

As is understood in the art, a gene comprises a coding strand and a non-coding strand. As used herein, the terms “coding strand”, “coding sequence” and “sense strand” are used interchangeably, and refer to a nucleic acid sequence that has the same sequence of nucleotides as an mRNA from which the gene product is translated. As is also understood in the art, when the coding strand and/or sense strand is used to refer to a DNA molecule, the coding/sense strand includes thymidine residues instead of the uridine residues found in the corresponding mRNA. Additionally, when used to refer to a DNA molecule, the coding/sense strand can also include additional elements not found in the mRNA including, but not limited to promoters, enhancers, and introns. Similarly, the terms “template strand” and “antisense strand” are used interchangeably and refer to a nucleic acid sequence that is complementary to the coding/sense strand.

Similarly, all genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Also encompassed are any and all nucleotide sequences that encode the disclosed amino acid sequences, including but not limited to those disclosed in the corresponding GENBANK® entries.

The term “gene expression” or “expression” generally refers to the cellular processes by which a biologically active polypeptide is produced from a DNA sequence and exhibits a biological activity in a cell. As such, gene expression involves the processes of transcription and translation, but also involves post-transcriptional and post-translational processes that can influence a biological activity of a gene or gene product, e.g. a polypeptide biomarker. These processes include, but are not limited to RNA syntheses, processing, and transport, as well as polypeptide synthesis, transport, and post-translational modification of polypeptides. Additionally, processes that affect protein-protein interactions within the cell can also affect gene expression as defined herein.

The terms “modulate” or “alter” are used interchangeably and refer to a change in the expression level of a gene, or a level of RNA molecule or equivalent RNA molecules encoding one or more proteins or protein subunits, or activity of one or more proteins or protein subunits is up regulated or down regulated, such that expression, level, or activity is greater than or less than that observed in the absence of the modulator. For example, the terms “modulate” and/or “alter” can mean “inhibit” or “suppress”, but the use of the words “modulate” and/or “alter” are not limited to this definition.

The terms “active”, “functional” and “physiological”, as used for example in “enzymatically active”, “functional chromatin” and “physiologically accurate”, and variations thereof, refer to the states of genes, regulatory components, chromatin, etc. that are reflective of the dynamic states of each as they exists naturally, or in vivo, in contrast to static or non-active states of each. Measurements, detections or screenings based on the active, functional and/or physiologically relevant states of biological indicators can be useful in elucidating a mechanism, or defining a disease state or phenotype, as it occurs naturally. This is in contrast to measurements taken based on static concentrations or quantities of a biological indicator that are not reflective of level of activity or function thereof.

As used herein, the terms “antibody” and “antibodies” refer to proteins comprising one or more polypeptides substantially encoded by immunoglobulin genes or fragments of immunoglobulin genes. The presently disclosed subject matter also includes functional equivalents of the antibodies of the presently disclosed subject matter. As used herein, the phrase “functional equivalent” as it refers to an antibody refers to a molecule that has binding characteristics that are comparable to those of a given antibody. In some embodiments, chimerized, humanized, and single chain antibodies, as well as fragments thereof, are considered functional equivalents of the corresponding antibodies upon which they are based. In some embodiments, the presently disclosed subject matter provides methods for identifying, characterizing and/or developing disease-related components of a gene-specific chromatin regulatory protein complex, wherein one or more antibodies can be used directly, or in assays related thereto, in the identification, characterization and/or isolation of such components.

The term “substantially identical”, as used herein to describe a degree of similarity between nucleotide sequences, peptide sequences and/or amino acid sequences refers to two or more sequences that have in one embodiment at least about least 60%, in another embodiment at least about 70%, in another embodiment at least about 80%, in another embodiment at least about 85%, in another embodiment at least about 90%, in another embodiment at least about 91%, in another embodiment at least about 92%, in another embodiment at least about 93%, in another embodiment at least about 94%, in another embodiment at least about 95%, in another embodiment at least about 96%, in another embodiment at least about 97%, in another embodiment at least about 98%, in another embodiment at least about 99%, in another embodiment about 90% to about 99%, and in another embodiment about 95% to about 99% nucleotide, peptide or amino acid identity, when compared and aligned for maximum correspondence, as measured using a sequence comparison algorithm or by visual inspection.

### II. Subjects

The subject screened, tested, or from which a sample is taken, is desirably a canine subject, although it is to be understood that the principles of the disclosed subject matter indicate that the compositions and methods are effective with respect to invertebrate and to all vertebrate species, including mammals, which are intended to be included in the term “subject”. A canine subject is any dog or dog-like mammal (i.e. members of the genus Canis, including dogs, wolves, coyotes, and jackals. A canine can in some embodiments preferably refer to the domestic dog.

In some embodiments, the subject to be used in accordance with the presently disclosed subject matter is a subject, e.g. a canine, in need of treatment and/or diagnosis. In some embodiments, a subject, particularly a canine subject, can have or be believed to be susceptible to CHD or related condition.

As used herein, the term “immature canine subject” refers to a canine that has not reached skeletal maturity, and/or is not an adult canine. An immature canine subject, or a canine that has not reached skeletal maturity, can be a canine that is less than about two years of age, less than about one year of age, or less than about six months of age.

### III. General Considerations

Provided herein are methods for predicting (or diagnosing or monitoring) canine hip dysplasia (CHD) in an immature canine subject (in some embodiments less than about two years of age, or less than about one year of age, or less than about six months of age). Such methods can comprise measuring in a biological sample from the immature canine subject the concentration of at least two polypeptides selected from the group consisting of: C2C, CTX-I, CTX-II, RANKL, PIICP, COMP, PINP, and fragments of any thereof, and any combination thereof, wherein the concentration of the at least two polypeptides or fragments thereof in the biological sample provide a sample protein profile indicative of the susceptibility to, presence or absence, degree, severity, type or stage of CHD in the immature canine subject.

In some embodiments, such methods of diagnosing and/or predicting CHD in a canine subject, particularly an immature canine, can comprise measuring biomarker concentration levels in a urine sample from the subject.

Also provided herein are canine hip dysplasia (CHD) biomarker profiles, such profiles comprising polypeptide concentration level information for two or more polypeptides selected from the group consisting of: C2C, CTX-I, CTX-II, RANKL, PIICP, COMP, PINP, and fragments of any thereof, and any combination thereof, obtained from a biological sample from an immature canine subject susceptible to having and/or developing CHD.

### IV. Detailed Description of Methods, Systems and Assays for Diagnosing and/or Predicting CHD

As discussed further herein, including the Examples section, the data of the present studies surprisingly revealed an ability to detect, predict and/or diagnose CHD in immature canines, in some cases well before symptoms of CHD were developed. Such unexpected findings provide meaningful advances of existing techniques to diagnose CHD, all of which rely upon the animal to have already developed the condition or progressed closed thereto, which limits the ability to be proactive in treating hip dysplasia and preventing or limiting secondary osteoarthritis.

Thus, in some embodiments, the presently disclosed subject matter is directed to biomarkers for early diagnosis of CHD and methods of using the same. Also provided are diagnostic and/or predictive kits and assays for CHD and related conditions in canines.

In some embodiments, provided are methods for predicting, diagnosing, monitoring and/or characterizing CHD in a canine subject, and particularly an immature canine subject. Such methods can include obtaining or securing a biological sample from the immature canine subject and measuring, detecting and/or quantifying one or more biological markers for CHD in the sample. More particularly, a concentration of at least two, three, four, five, six or seven polypeptide biomarkers, or concentration of at least two, three, four, five, six or seven polypeptide biomarkers, can be measured in the sample, where the polypeptides are selected from C2C, CTX-I, CTX-II, RANKL, PIICP, COMP, PINP. Based on the concentration and/or expression level of the at least two polypeptides, i.e. two, three, four, five, six or seven polypeptides, a polypeptide profile or biomarker concentration profile can be established. Such a polypeptide profile or biomarker concentration profile can be used as a signature to diagnose or predict CHD in other canine subjects. For example, an elevated level of the at least two polypeptides in a biological sample, as compared to a control, can provide a sample polypeptide profile, biomarker concentration profile, or signature that is indicative of a susceptibility to CHD, particularly as compared to an unelevated level of production of the at least two polypeptides in the biological sample from a control (i.e. from a subject not susceptible to CHD). For example, reduced or decreased level of expression of the at least two polypeptides in a biological sample, as compared to a control, can provide a sample polypeptide profile, biomarker concentration profile, or signature that is indicative of a susceptibility to CHD, particularly as compared to a normal or control concentration of the at least two polypeptides in the biological sample from a control (i.e. from a subject not susceptible to CHD). The sample polypeptide profile, biomarker concentration profile, or signature can be based on an elevated, reduced, or combination of both, concentration or production of any combination and number of C2C, CTX-I, CTX-II, RANKL, PIICP, COMP, PINP. As the data herein shows, it is these polypeptide concentration or production profiles that allow for the prediction and/or diagnosis of CHD in the immature canine subjects, which has never before been achieved.

By way of example and not limitation, in some embodiments, an immature canine can be considered to have elevated levels of biomarkers in urine samples when C2C is above 0.82 ng/mL, COMP is above 0.21 ng/mL, CTX-1 is above 0.07 ng/mL, CTX-II is above 9.75 pg/mL, RANKL is above 0.22 ng/mL, PIICP is above 0.25 ng/mL, and PINP is above 7.58 pg/mL. In some embodiments, 5 month old male puppies are considered to have elevated levels of biomarkers in serum samples when C2C is above 24.40 ng/mL, COMP is above 3.47 ng/mL, CTX-I is above 4.99 ng/mL, CTX-II is above 506.24 pg/mL, RANKL is above 13.58 ng/mL, PIICP is above 20.64 ng/mL, and PINP is above 305.86 pg/mL. In some embodiments, such changes, e.g. elevated or decreased concentrations, can be about 10% to more than 100% as compared to a control, optionally about 5%, 10%, 15%, 20%, 25%, 30%, 40%, 50% or more increased or decreased as compared to a control.

In some embodiments, such methods, including methods of diagnosing and/or predicting CHD, can further comprise a treatment and/preventative step. For example, by using the disclosed methods breeders can be better informed so as to discourage breeding of CHD bloodlines to avoid propagating the CHD problem in future generations. Additionally, in some embodiments the methods can further comprise treating the subject diagnosed with CHD by administering any suitable form of treatment for CHD, including but not limited to administering physical therapy for hip strengthening to reduce laxity in canine patient, and/or by performing a surgery (e.g. in juvenile canines pubic symphysiodesis or pelvic osteotomies can be performed). Additionally, in some embodiments the methods can further comprise treating the subject diagnosed with CHD by redirecting dog training (pet versus a performance, military, and service dogs) and expectations (pet level dog versus performance level dog). Currently, performance, military, and service dogs can cost $200K or more to get them to their peak level and there is no way to know of a CHD problem before investing all of that money, so a diagnostic method and/or test as disclosed herein can provide helpful insight.

In some embodiments, the immature canine subjects in the disclosed methods include canines that have not reached skeletal maturity, or are less than about two years of age, optionally less than about one year of age, or less than about six months of age.

The biological samples suitable for such methods and assays include any of urine, whole blood, blood plasma, synovial fluid and serum. In some preferred embodiments the biological sample can be urine. The ability to use urine samples can be advantageous for its ease of collection, processing and storage.

More particularly, in some embodiments the concentration of at least three polypeptides selected from the group consisting of C2C, CTX-I, CTX-II, RANKL, PIICP, COMP and PINP can be measured, wherein the sample polypeptide concentration profile is based on at least three polypeptides in the biological sample. An elevated or decreased (or combination) concentration of at least two of the three polypeptides, or in some embodiments all three, in the biological sample, as compared to a control, can thus provide a sample polypeptide profile indicative of a susceptibility to CHD.

Correspondingly, the concentration of at least four polypeptides selected from the group consisting of C2C, CTX-I, CTX-II, RANKL, PIICP, COMP and PINP is measured, wherein the sample polypeptide concentration profile can be based on at least four polypeptides in the biological sample. An elevated or decreased (or combination of elevated and decreased) concentration of at least three of the four polypeptides, or in some embodiments all four, in the biological sample, as compared to a control, provides a sample polypeptide profile indicative of a susceptibility to CHD.

In some embodiments, the concentration of each of C2C, CTX-I, CTX-II and RANKL can be measured in a urine sample from an immature canine subject, wherein the sample polypeptide concentration or production profile can be based on the concentration of each of C2C, CTX-I, CTX-II and RANKL in the urine sample. Alternatively, or in addition, the concentration of each of C2C, COMP, PIICP, CTX-II and RANKL can be measured in a serum sample from an immature canine subject, wherein the sample polypeptide concentration profile can be based on the concentration of each of C2C, COMP, PIICP, CTX-II and RANKL in the serum sample.

Provided herein are CHD biomarker concentration or production profiles, such profiles being useful as biomarkers or signatures for diagnostic and predictive applications, particularly in immature or young canines. Such a CHD biomarker profile can comprise polypeptide concentration levels for two or more polypeptides selected from the group consisting of: C2C, CTX-I, CTX-II, RANKL, PIICP, COMP, PINP, and fragments of any thereof, and any combination thereof, obtained from a biological sample from at least one immature canine subject susceptible to having and/or developing CHD.

Of note, fragments of any of C2C, CTX-I, CTX-II, RANKL, PIICP, COMP, PINP can comprise polypeptides, or polynucleotides encoding the same, that are substantially similar, substantially identical to, or substantially complete portions thereof. For example, fragments of each biomarker that are “substantially identical” to the full length or native biomarker can be described as those having a degree of similarity to the native biomarker's nucleotide sequences, peptide sequences and/or amino acid sequences in one embodiment at least about least 60%, in another embodiment at least about 70%, in another embodiment at least about 80%, in another embodiment at least about 85%, in another embodiment at least about 90%, in another embodiment at least about 91%, in another embodiment at least about 92%, in another embodiment at least about 93%, in another embodiment at least about 94%, in another embodiment at least about 95%, in another embodiment at least about 96%, in another embodiment at least about 97%, in another embodiment at least about 98%, in another embodiment at least about 99%, in another embodiment about 90% to about 99%, and in another embodiment about 95% to about 99%, when compared and aligned for maximum correspondence, as measured using a sequence comparison algorithm or by visual inspection.

The CHD biomarker concentration profiles can further comprise polypeptide concentration levels for at least one biological sample obtained from at least one healthy canine subject and/or a canine subject not susceptible to developing CHD. The biomarker concentration profiles can be based on measurements taken from samples of urine, whole blood, blood plasma, synovial fluid and/or serum. In some embodiments, urine samples can be advantageously used. By way of example and not limitation, the CHD biomarker concentration profiles can be based on polypeptide concentration levels for three or more polypeptides selected from the group consisting of C2C, CTX-I, CTX-II, RANKL, PIICP, COMP, PINP, or four or more polypeptides selected from the same. Alternatively, such CHD biomarker concentration profiles can comprise polypeptide concentration levels of each of C2C, CTX-I, CTX-II and RANKL, wherein the sample is a urine sample. Additionally, in some embodiments CHD biomarker concentration profiles can comprise polypeptide concentration levels of each of C2C, COMP, PIICP, CTX-II and RANKL, wherein the sample is a serum sample.

In some embodiments, provided herein are diagnostic reagents for predicting canine hip dysplasia (CHD) comprising at least one antibody against at least one CHD biomarker or fragment thereof selected from the group consisting of: C2C, CTX-I, CTX-II, RANKL, PIICP, COMP, PINP. The diagnostic reagent can comprise at least two or more antibodies (Ahner et al. 2019 paper in J Orthop Res 37:916-920) against any two or more CHD biomarkers or fragments thereof. Kits comprising such diagnostic reagents are also provided.

In some aspects, provided herein are kits for diagnosing and/or predicting CHD in an immature canine subject. Such kits can comprise at least one CHD biomarker detection reagent that specifically binds to a CHD polypeptide selected from the group consisting of C2C, CTX-I, CTX-II, RANKL, PIICP, COMP, PINP and fragments of any thereof, or at least one CHD biomarker detection reagent that specifically binds to at least part of a polynucleotide sequence coding for at least one of the CHD polypeptides, wherein the specific binding of the reagent is indicative of the concentration level of at least one of the CHD polypeptides in a biological sample from a subject. Such kits can further comprise at least two CHD biomarker detection reagents that each specifically binds to a CHD polypeptide, or at least two CHD biomarker detection reagents that each specifically binds to at least part of a polynucleotide sequence coding for at least one of the CHD polypeptides.

In each of the above methods, reagents, kits and the like the concentration of one or more of the biomarkers is measured in a sample collected from a subject to be screened or tested. In measuring the concentration of any of the urine or plasma biomarkers, e.g. C2C, CTX-I, CTX-II, RANKL (GenBank: AAB86811.1), PIICP, COMP (NCBI Reference Sequence: XP_038284037.1), PINP, assays (some of which are commercially available, e.g. from NeoBio (Neo Scientific, Cambridge, Massachusetts) and/or MyBioSource (MyBioSource, Inc., San Diego, California) can measure the entire protein or peptide of the biomarker, or a fragment or portion thereof (Note CTX-II, CTX-I, C2C, PIICP, and PINP are protein fragments from the degradation (CTX-II, C2C, and CTX-I) of collagen type II or type I, or processing of the pro-collagen protein during synthesis). Further details regarding C2C, CTX-I, CTX-II, RANKL, PIICP, COMP, PINP can be found in Elsaid & Chichester (Clinica Chimica Acta 365 (2006) 68-77), Melkko et al. (Clinical Chemistry 42:6 (1996) 947-954, and Herman & Seibel (Clinica Chimica Acta 393 (2008) 57-75). Any suitable assay for detecting and/or measuring/quantifying the peptide biomarkers can be used and is within the scope of the present disclosure, including but not limited to commercially available enzyme-linked immunosorbent assays (ELISA) that are configured to quantify C2C, CTX-I, CTX-II, RANKL, PIICP, COMP, PINP, and fragments of each. Other such assays include, but are not limited to LUMINEX, ELISA, immunoassay, mass spectrometry, high performance liquid chromatography, two-dimensional electrophoresis, in situ hybridization, SAGE, Western blotting, protein microarray, and antibody microarray.

### EXAMPLES

The following examples are included to further illustrate various embodiments of the presently disclosed subject matter. However, those of ordinary skill in the art should, in light of the present disclosure, appreciate that many changes can be made in the specific embodiments which are disclosed and still obtain a like or similar result without departing from the spirit and scope of the presently disclosed subject matter.

### Example 1

### Serum and Urine Protein Biomarkers for Early Diagnosis of Hip Dysplasia

An experiment was designed to identify protein biomarkers with high discriminatory capabilities for differentiating dogs with dysplastic hips from non-dysplastic dogs at an early age, i.e., prior to skeletal maturity, or less than about 1 year of age.

With IACUC approval, informed client consent was obtained from owners of 8- to 12-week-old puppies of various breeds recruited from across the United States. Based on these criteria 14 large breed dogs (8 males and 6 females) were enrolled in an initial study. Radiographs were evaluated for joint pathology based on Orthopedic Foundation for Animals (OFA) grading criteria (hip dysplasia diagnosed at 2 years of age: 3 males, 1 female).

Whole blood and urine were collected on the same day at 8 time points (3, 4, 5, 6, 9, 12, 18 to 24 months of age) and then shipped to a laboratory overnight on ice. For processing, 5-10 mL of each coagulated blood sample was centrifuged at 20,000 rpm for 20 min to obtain serum. The serum and urine were aliquoted into separate tubes and stored at −80° C. until analysis.

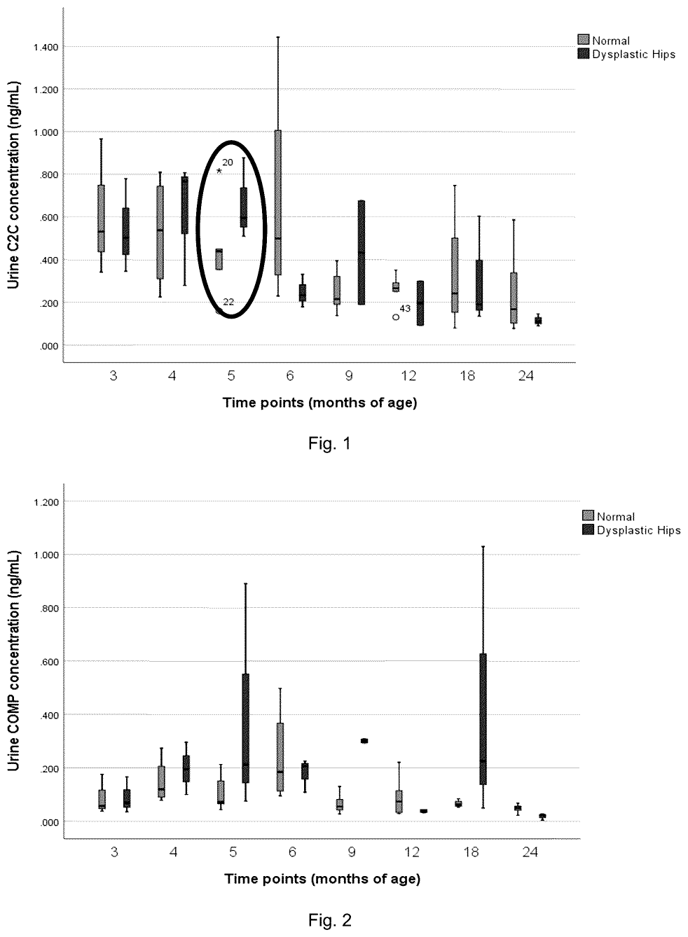

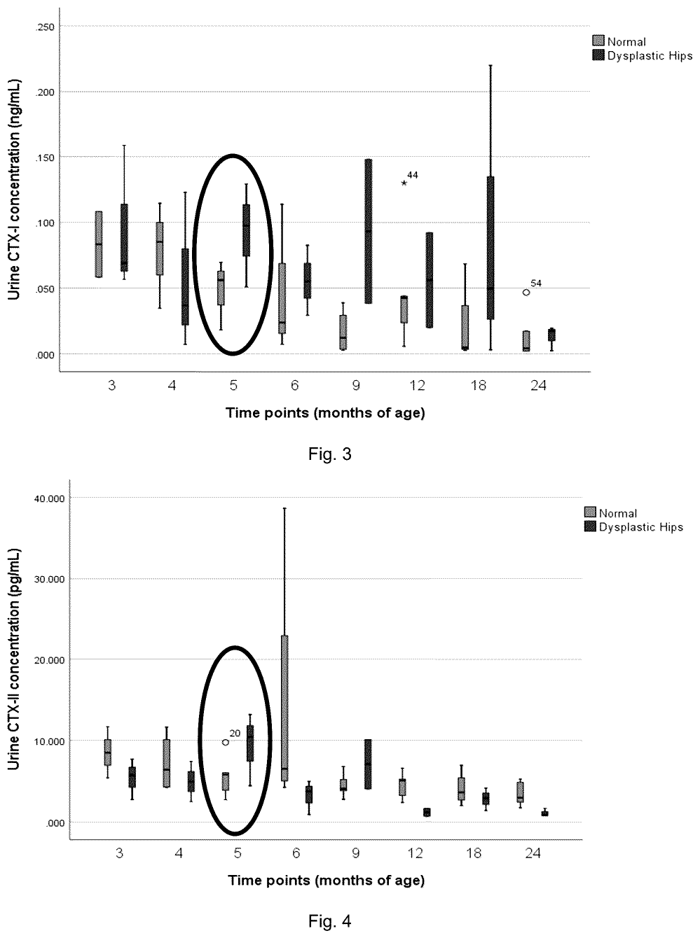

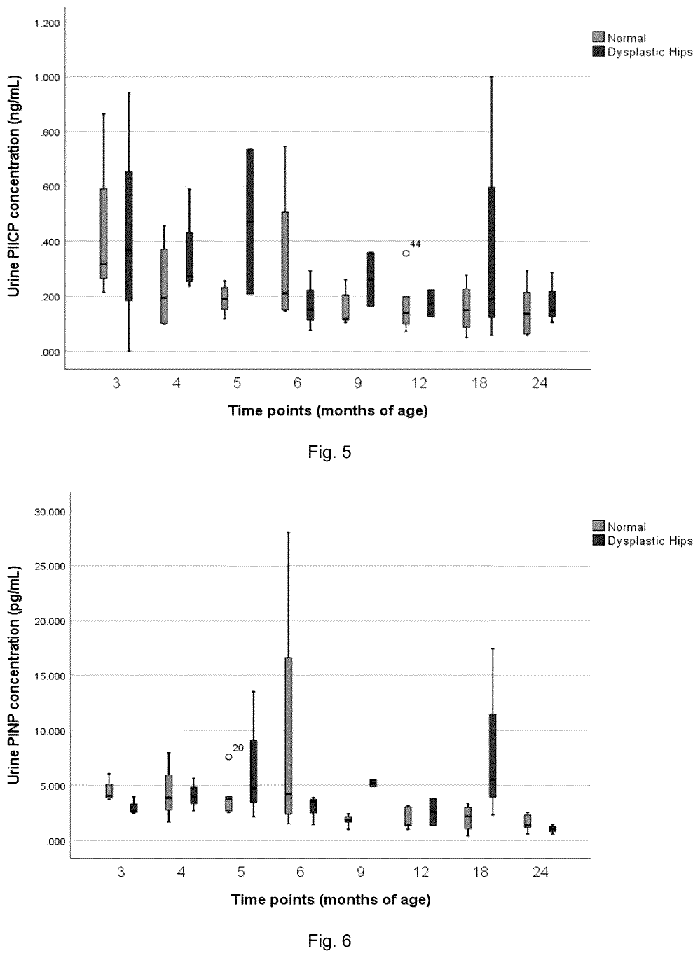

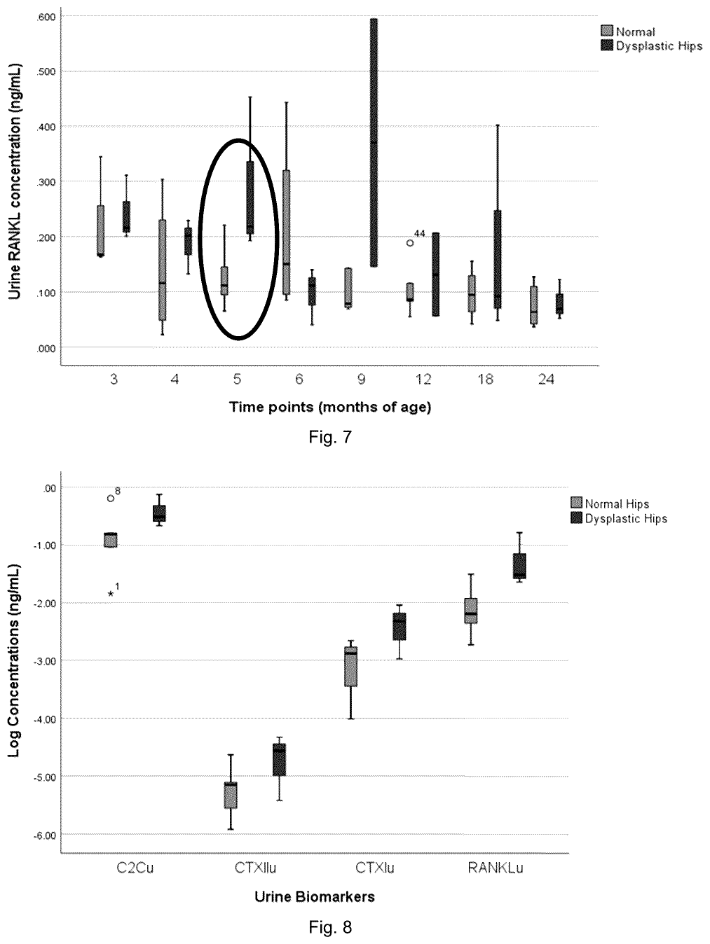

Seven potential biomarkers believed to reflect direct and/or indirect measures of joint health were measured in serum and urine using commercially available enzyme-linked immunosorbent assays (ELISA) according to the manufacturer's instructions (except the sample and enzyme solution were incubated overnight at 4° C. to limit nonspecific binding). The biomarkers tested and/or screened for included the following: cartilage oligomeric matrix protein (COMP), cross linked C-telopeptide of type I, II collagen (CTX-I, CTX-II), receptor activator of nuclear factor kappa-B ligand (RANKL), procollagen type I N-terminal propeptide (PINP), collagenase-generated cleavage epitope of type II collagen (C2C), and procollagen type II C-terminal propeptide (PIICP). See FIGS. 1-7 (urine) and 9-15 (serum). These assays were validated to cross-react with samples of canine origin. The urine creatinine concentration was measured with a creatinine colorimetric assay and used to standardize the urinary concentrations obtained for the other assays.

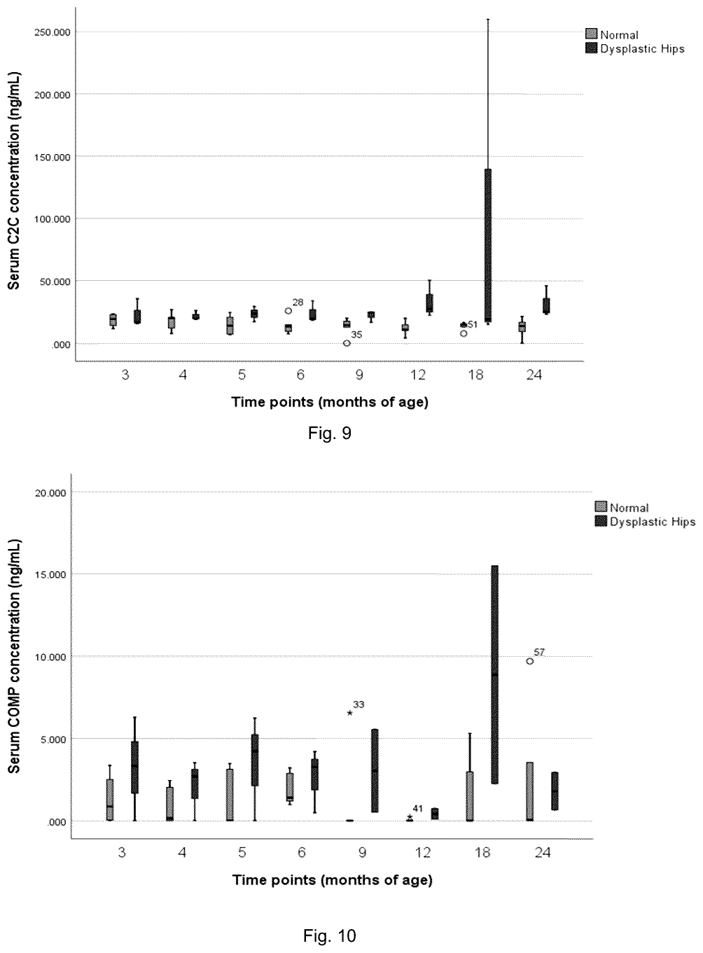

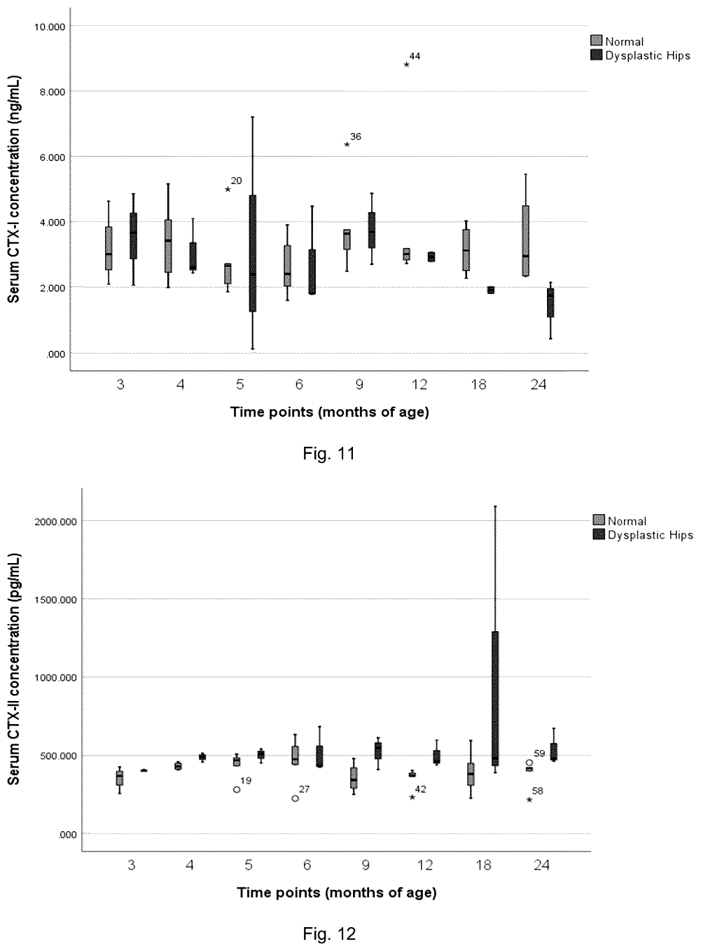

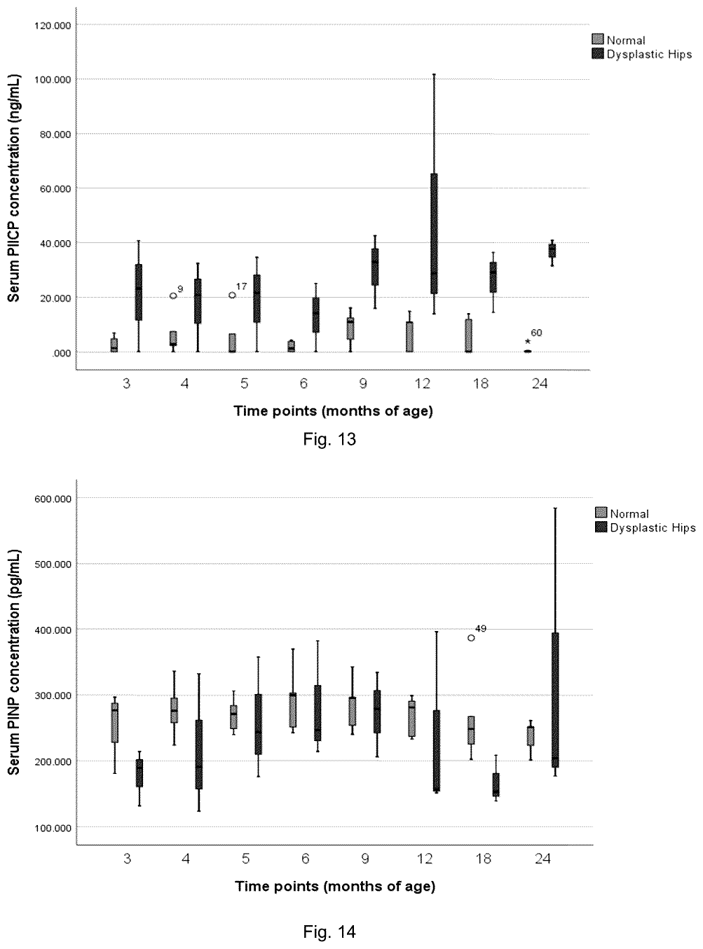

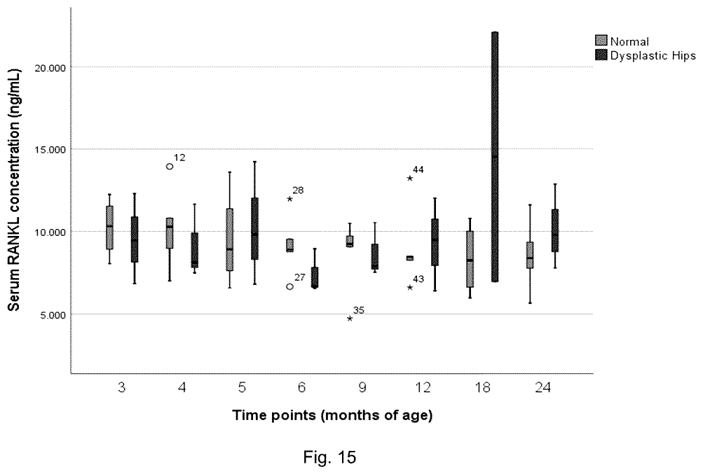

At 5 months of age, immature canines showed marked differences in concentrations of several targeted urine biomarkers (C2C, CTX-I, CTX-II, RANKL) and serum PIICP between dysplastic and non-dysplastic hip cohorts. At 5 months of age, immature dogs with dysplastic hips had higher levels of all targeted urine biomarkers, higher levels of serum C2C, COMP, PIICP, CTX-II and RANKL, and lower levels of CTX-I and PINP as compared to males with normal hips (See FIGS. 1-7 and 9-15). Females exhibited a variability in biomarker concentrations throughout the study with peak levels commonly seen at 5-6 months and 12-24 months in intact females.

### DISCUSSION OF RESULTS

Based on the data provided in Example 1, urine biomarkers, and kits and methods based thereon, can unexpectedly provide early, accurate diagnosis of CHD in canines, particularly immature canines. Particularly, as shown in FIG. 8, at least urine biomarkers C2C, CTX-II, CTX-I, and RANKL were increased in immature canines at 5 months of age that would later develop hip dysplasia. Unlike prior findings, these data are the first to identify biomarkers, including panels of biomarkers, that are suitable for predicting, diagnosing or monitoring CHD in immature canines. Importantly, this allows for the ability to diagnose and/or predict CHD at an early age such that therapies and preventive measures can be taken.

These unexpected findings have high impact and translational potential based on the relative ease and non-invasive nature of urine collection, the ability to preserve and ship samples effectively, and the availability and efficiency of the analysis. The use of these targeted biomarkers provides a method for disease screening, early diagnosis and treatment monitoring in CHD, as well as related conditions, e.g. developmental dysplasia of the hip (DDH), in humans.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method for diagnosing canine hip dysplasia (CHD) in an immature canine subject, the method comprising:
obtaining a biological sample from an immature canine subject;
measuring in the biological sample from the immature canine subject a concentration of at least two polypeptides selected from the group consisting of: C2C, CTX-I, CTX-II, RANKL, PIICP, COMP, PINP;
generating a sample polypeptide concentration profile based on the concentration of the at least two polypeptides in the biological sample, wherein an elevated or decreased concentration of the at least two polypeptides in the biological sample, as compared to a control, provides a sample polypeptide concentration profile indicative of CHD, wherein a concentration of the at least two polypeptides in the biological sample that is not elevated or decreased, as compared to a control, provides a sample polypeptide concentration profile not indicative of CHD; and using the generated sample polypeptide concentration profile to diagnose CHD in the immature canine subject.

2. The method of claim 1, wherein the immature canine subject is a canine that has not reached skeletal maturity or is less than about one year of age.

3. The method of claim 1, wherein the immature canine subject is a canine that is less than about six months of age.

4. The method of claim 1, wherein the biological sample comprises any one of urine, whole blood, blood plasma, synovial fluid and serum.

5. The method of claim 1, wherein the biological sample comprises urine.

6. The method of claim 1, wherein the concentration of at least three polypeptides selected from the group consisting of C2C, CTX-I, CTX-II, RANKL, PIICP, COMP and PINP is measured, and wherein the sample polypeptide concentration profile is based on the concentration of the at least three polypeptides in the biological sample.

7. The method of claim 6, wherein an elevated or decreased concentration of at least two of the three polypeptides in the biological sample, as compared to a control, provides a sample polypeptide concentration profile indicative of CHD.

8. The method of claim 6, wherein an elevated or decreased concentration of the three polypeptides in the biological sample, as compared to a control, provides a sample polypeptide concentration profile indicative of CHD.

9. The method of claim 1, wherein the concentration of at least four polypeptides selected from the group consisting of C2C, CTX-I, CTX-II, RANKL, PIICP, COMP and PINP is measured, and wherein the sample polypeptide concentration profile is based on the concentration of the at least four polypeptides in the biological sample.

10. The method of claim 9, wherein an elevated or decreased concentration of at least three of the four polypeptides in the biological sample, as compared to a control, provides a sample polypeptide concentration profile indicative of CHD.

11. The method of claim 9, wherein an elevated or decreased concentration of the four polypeptides in the biological sample, as compared to a control, provides a sample polypeptide concentration profile indicative of CHD.

12. The method of claim 1, wherein the concentration of each of C2C, CTX-I, CTX-II and RANKL is measured in a urine sample from the immature canine subject, and wherein the sample polypeptide concentration profile is based on the concentration of each of C2C, CTX-I, CTX-II and RANKL in the urine sample.

13. The method of claim 1, wherein the concentration of each of C2C, COMP, PIICP, CTX-II and RANKL is measured in a serum sample from the immature canine subject, and wherein the sample polypeptide concentration profile is based on the concentration of each of C2C, COMP, PIICP, CTX-II and RANKL in the serum sample.

14. The method of claim 1, wherein the concentration of the at least two polypeptides in the biological sample is measured using a method selected from the group consisting of: LUMINEX, ELISA, immunoassay, mass spectrometry, high performance liquid chromatography, two-dimensional electrophoresis, in situ hybridization, SAGE, Western blotting, protein microarray, and antibody microarray.

15. A canine hip dysplasia (CHD) biomarker profile comprising polypeptide concentration levels for two or more polypeptides selected from the group consisting of: C2C, CTX-I, CTX-II, RANKL, PIICP, COMP, PINP, and fragments (e.g. portion of the full protein structure) of any thereof, and any combination thereof, obtained from a biological sample from at least one immature canine subject susceptible to having and/or developing CHD.

16. The CHD biomarker profile of claim 15, further comprising polypeptide concentration levels for at least one biological sample obtained from at least one healthy canine subject and/or a canine subject not susceptible to developing CHD.

17. The CHD biomarker profile of claim 16, wherein the biological samples from the immature canine subject susceptible to having and/or developing CHD and the healthy subject and/or the subject not susceptible to developing CHD both comprise a sample selected from the group consisting of urine, whole blood, blood plasma and serum.

18. The CHD biomarker profile of claim 17, wherein the biological samples are urine sample.

19. The CHD biomarker profile of claim 15, comprising polypeptide concentration levels for three or more polypeptides selected from the group consisting of C2C, CTX-I, CTX-II, RANKL, PIICP, COMP, PINP.

20. The CHD biomarker profile of claim 15, comprising polypeptide concentration levels for four or more polypeptides selected from the group consisting of C2C, CTX-I, CTX-II, RANKL, PIICP, COMP, PINP.

21. The CHD biomarker profile of claim 15, comprising polypeptide concentration levels of each of C2C, CTX-I, CTX-II and RANKL, wherein the sample is a urine sample.

22. The CHD biomarker profile of claim 15, comprising polypeptide concentration levels of each of C2C, COMP, PIICP, CTX-II and RANKL, wherein the sample is a serum sample.
